(19) United States
(12) Patent Application Publication
(10) Pub. No.: US 2024/0342408 A1
(43) Pub. Date: Oct. 17, 2024

(54) LIQUID DISPENSER

(71) Applicant: Aptar Radolfzell GmbH, Radolfzell (DE)

(72) Inventor: Leonard WELSCH, Karlsruhe (DE)

(21) Appl. No.: 18/634,022

(22) Filed: Apr. 12, 2024

(30) Foreign Application Priority Data
Apr. 14, 2023 (EP) 23168076.0

(51) Int. Cl. A61M 15/08; A24F 42/20; A24F 42/60; A61M 11/00; A61M 11/04; A61M 15/00; A61M 15/06; B65D 83/28

(52) U.S. Cl. CPC A61M 15/08; A24F 42/20; A24F 42/60; A61M 11/006; A61M 11/04; A61M 15/009; A61M 15/06; B65D 83/28; A61M 2205/123

(57) ABSTRACT

A liquid dispenser having a liquid reservoir, a liquid applicator and a conveying device for conveying liquid from the liquid reservoir) to the liquid applicator. The liquid dispenser has an applicator unit, on which the liquid applicator is provided, and a protective unit having a protective cover for protecting the applicator unit. The protective unit and the applicator unit can be displaced with respect to one another between a protective position and a use position. The liquid dispenser has an operating handle, via which operating handle the protective unit can be displaced from the protective position into the use position with respect to the applicator unit, and by which, in the use position, the conveying device can be actuated and liquid can be conveyed from the liquid reservoir to the liquid applicator.

### CROSS-REFERENCE TO RELATED APPLICATION

This claims priority from European Application No. 23168076.0, filed Apr. 14, 2023, the disclosure of which is hereby incorporated by reference in its entirety.

### TECHNICAL FIELD

The invention relates to a conventionally portable liquid dispenser, by means of which liquids are discharged, preferably in atomized form. The design of a liquid dispenser according to the invention is suitable for various medical applications, but also for non-medical applications such as discharging liquids containing nicotine.

### BACKGROUND AND SUMMARY

In the case of liquid dispensers that are used frequently by the user thereof, for example liquid dispensers for discharging nicotine-containing or cannabis-containing products, or analgesics, it is desired for these to be able to be operated in as comfortable a manner as possible, i.e. to be able to be utilized by way of a straightforward and habitual hand movement. It is however also desired for the liquid dispensers for this purpose to initially be transferred into a use state in which the discharging can then take place. This requirement of the initial transfer into a use state is intended to prevent the liquid dispenser from unexpectedly releasing liquid or from being too easy for small children to operate.

The invention provides a liquid dispenser that enables straightforward operation, but at the same time can be configured in such a manner that it effectively prevents accidental actuations and/or actuation by small children.

In order to achieve this, a liquid dispenser is proposed that has a liquid reservoir and a liquid applicator and also a conveying device for conveying liquid from the liquid reservoir to the liquid applicator. The liquid can be discharged at the liquid applicator, for which purpose at least one discharge opening is provided.

The conveying device is preferably a pump device, which draws in liquid from an unpressurized liquid reservoir and discharges it. Alternatively, the conveying device can however also be formed by a simple outlet valve, which allows liquid that has previously been stored under pressure to escape from a liquid reservoir designed as a pressurized reservoir, as soon as the valve is opened.

The liquid dispenser is used in particular to discharge liquid in atomized form, wherein this is preferably achieved by a spray geometry with a vortex chamber connected upstream of the discharge opening or by a nozzle unit with a plurality of fine nozzle openings. Medical and non-medical fields of application are provided for a liquid dispenser of the kind described here. In particular for medical applications, a configuration is provided in which the liquid applicator is designed in the manner of a nasal applicator with an elongate and tapering form. In particular for non-medical applications, specifically for the consumption of liquids containing nicotine or cannabis, the configuration with a cylindrical oral applicator is provided.

The liquid dispenser is designed as a portable liquid dispenser, which a user conventionally carries with them. The liquid reservoir therefore has a small volume. It is preferably less than 50 ml, particularly preferably less than 10 ml. Its length is preferably less than 150 mm, in particular less than 120 mm.

As a whole, the liquid dispenser preferably has an elongate form. Along a longitudinal axis, it is preferably larger by a factor of 2 than in the transverse direction thereto. The longitudinal sides of the liquid dispenser are used in particular for gripping in one hand. The liquid applicator is preferably provided on an end side of the liquid dispenser.

In order to protect the liquid applicator of the liquid dispenser in the carried state, the liquid dispenser has an applicator unit, on which the liquid applicator is provided, and a protective unit with a protective cover for protecting the applicator unit. The protective unit is a non-detachable part of the liquid dispenser. During normal use of the liquid dispenser, said protective unit remains constantly connected to the applicator unit.

The protective unit and the applicator unit can be displaced with respect to one another, preferably in a linear manner and along a main axis of the liquid dispenser, between a protective position and a use position. In the protective position, the protective unit at least partially surrounds the liquid applicator and as a result protects it. In the use position, which can be achieved by manual relative displacement, the liquid applicator is in an exposed position relative to the protective unit and is at least partially displaced out of the protective cover. In this position, it is now possible to discharge liquid. The distance by which the applicator unit and the protective unit can be displaced with respect to one another is preferably at least 5 mm, particularly preferably at least 10 mm, or even at least 15 mm.

An operating handle, which is able to move with respect to the applicator unit and with respect to the protective unit, is provided both for the relative displacement of the protective unit and the applicator unit with respect to one another and for achieving the discharging of liquid itself. Said operating handle can be able to move in a linear manner, but is preferably installed as a pivotable operating lever that is able to pivot with respect to the protective unit and the applicator unit. The operating handle is preferably displaced at least in phases in a direction that is at an angle to the relative displacement direction of the protective unit and applicator unit, preferably at an angle by an angle of at least 75°.

The protective unit can be displaced with respect to the applicator unit from the protective position into the use position by means of the operating handle. The operating handle is accordingly installed and configured such that it enables the force required for the relative displacement from the protective position into the use position to be coupled onto the protective unit and the applicator unit. The operating handle is preferably provided on a longitudinal side of the liquid dispenser. Usage is intended to take place in such a manner that the user grips the applicator unit and applies a force to the operating handle with one finger or in particular the thumb, in order to transfer the protective unit into the use position, and immediately afterwards applies a force to the operating handle once again, in order to cause liquid to be discharged, by virtue of the conveying device being actuated by means of the operating handle and liquid being conveyed from the liquid reservoir to the liquid applicator.

The described design of the liquid dispenser enables very comfortable operation, but at the same time is highly suitable for providing protection against accidental transfer into the use position or even unintended discharging of liquid. The ability to use the operating handle for establishing the use state and for discharging liquid enables both to be achieved in one movement.

Although comfortable operation is aimed for in the liquid dispenser according to the invention, the liquid dispenser preferably has a blocking device, which has to be released during usage in order to enable the protective unit and the applicator unit to be displaced from the protective position into the use position. The blocking device can preferably also be released by means of the operating handle, so that the protective unit and the applicator unit can then be displaced into the use position.

The purpose of the blocking device is to prevent the liquid dispenser from inadvertently being transferred into the use position. This firstly applies to inadvertent transfer caused by unintended application of force in a pocket or the like. Depending on the configuration, a blocking device can however also be used as a child lock, which at least effectively prevents small children from establishing the use state.

The blocking device is preferably designed in such a manner that the protective unit can only begin to be displaced to the relevant extent with respect to the applicator unit once the operating handle has been actuated beforehand. In order to use the liquid reservoir, the operating handle thus has to be initially displaced, in order to make it possible for the protective unit to move as a result. Once the use position has been reached, a further force is applied to the operating handle for the purpose of discharging liquid. Mutually opposing movements of the operating handle are preferably provided for releasing the blocking device and for achieving the discharging of liquid.

The operating handle is preferably coupled to the blocking device in such a manner that the blocking device can be released by the operating handle being displaced outwards and in particular in a direction pointing away from the liquid applicator. This results in the overall movement for releasing the blocking and for establishing the use state comprising a first partial movement of the operating handle in a first direction for releasing the blocking and a second partial movement of the operating handle in a different, second direction for displacing the protective unit. The sequence is difficult for small children to carry out, in particular when the first partial movement takes place counter to a spring force and thus gripping is made more difficult for the child for the second partial movement, since the operating handle reverts to its blocking starting position when released.

The operating handle is also used as described for the application of force to the applicator unit and the protective unit with respect to one another. Even though the operating handle is able to move with respect to the two units, it can however preferably be displaced together with the protective unit with respect to the applicator unit. The operating handle is thus coupled to the protective unit in relation to the displacement direction in such a manner that force transmission is possible in the displacement direction.

In particular, a slot guide, by means of which the operating handle is guided movably on the protective unit, can be provided for this purpose. The slot guide makes it simultaneously possible for the operating handle to be able to move with respect to the protective unit and to be able to move together with the protective unit with respect to the applicator unit. The slot guide preferably extends orthogonally to the displacement direction of the protective unit and of the applicator unit, but forms at least an angle of 70° therewith.

As already explained, it is preferable if the pivotable operating lever is mounted on the applicator unit, which is provided on an end of the operating lever that points away from the liquid applicator, so as to be able to pivot about a pivot pin. This means that, after the use position has been reached, the lever is pivoted about a pivot pin that is substantially fixed in position in relation to the applicator unit. The opposite, pivoting end of the operating lever points towards the end of the liquid dispenser on which the liquid applicator and the protective cover are provided.

The pivotable operating lever will preferably be pivoted, starting from a rest position, outwards and/or away from the nasal applicator, in order to release the abovementioned blocking device. In order to be able to pivot the operating lever outwards, a grip elevation, which extends over an end side of the liquid dispenser and is easy to grasp, is preferably provided at a distal end of the operating lever.

The pivotable operating lever will preferably be pivoted, starting from a pivoted-out position, inwards and/or towards the nasal applicator, in order to actuate the conveying device. In particular, the operating lever can apply the inward movement, in particular via a deflection gear, to an outlet valve or a pump piston of the conveying device.

If the operating lever is fastened to the protective unit in relation to the displacement direction of the applicator unit and of the protective unit, as described above, then the pivot pin of said operating lever is displaced relative to the applicator unit during the displacement. For this purpose, the pivot pin is preferably fastened so as to be movably guided on the applicator unit. In particular, provision can be made for a slot guide, by means of which the movement of the pivot pin with respect to the applicator unit is controlled. The slot guide is preferably provided on a housing of the applicator unit.

In a particularly advantageous configuration, the guide has a direction of extent that, at least in certain sections, deviates from the displacement direction of the protective unit with respect to the applicator unit. This results in the pivot pin of the operating lever, when the protective unit is transferred from the protective position into the use position, being displaced so as to deviate from the displacement direction. This can result in the end of the operating lever penetrating into the housing of the applicator unit. This makes it possible to avoid the need to provide an opening that is free in the protective position on the applicator unit in order to receive the operating lever after transfer into the use position. Since it is preferable for the protective portion, the applicator portion and the operating lever together to form a body shape that is closed in the protective position when not being used and avoids the ingress of dirt or leakage of liquid, it is helpful to avoid an external opening for the operating lever.

The already mentioned blocking device can be realized in various ways, wherein it is preferable for the two independent partial devices described below to be realized together.

The blocking device can in particular comprise a protective panel, which can be displaced by means of the operating handle from a position in aligned arrangement in relation to the liquid applicator and into a position that is offset therefrom. In the aligned arrangement, the protective panel prevents the transfer into the use state, since the liquid applicator cannot be extended and would instead collide with the protective panel.

The abovementioned aperture can be closable using the abovementioned protective panel. For this purpose, the protective panel can be displaceable between the open position and the closed position, in particular in a guide of the protective unit.

In particular, the protective unit has an end wall provided with an aperture. The liquid applicator is pushed out through this aperture in the use position. In the protective position, by contrast, the liquid applicator is retracted and entirely within an interior space delimited by the end wall. This isolation of the liquid applicator in the abovementioned interior space in the protective position is advantageous, in particular in liquid dispensers that have a nozzle unit with a plurality of fine nozzle openings for atomizing the liquid. The risk posed by contamination is particularly relevant here. The isolation prevents or reduces contamination.

In the preferred configuration of the nozzle unit, it preferably has at least 10 nozzle openings with a minimum clear cross section of at most 0.02 mm² in each case. Preferably, at least 25 nozzle openings are provided.

The protective panel can be displaced into the open position by means of the operating handle, so that it is then possible to extend the liquid applicator. In this case, the protective panel is preferably only coupled to the operating handle in a unidirectional manner, so that the protective panel can be displaced into the open position but can remain in the open position if the operating handle is moved in the opposite direction. This avoids the protective panel colliding with the liquid applicator when the operating handle is actuated for the purpose of discharging liquid and, as a result, prevents continued displacement of the operating handle.

The protective panel is preferably assigned a spring means, which constantly applies a force in the direction of the closed position to the protective panel. As soon as the liquid dispenser has been brought from the use position into the protective position, this spring means presses the protective panel into the closed position and thus re-establishes the blocking and also closes the aperture, which is optionally provided, in the end wall.

Another form of blocking device can be provided in the region of the operating handle itself. The operating handle and the applicator unit can have abutment surfaces, which, in the protective position and when the operating handle is pressed in or in particular when the operating lever is pivoted in, prevent the protective unit from moving with respect to the applicator unit. When the operating lever is pivoted out or the operating handle is pulled out, the abutment surfaces disengage and enable the protective unit to be displaced.

The abutment surface of the operating handle is preferably provided in a first recess of the operating handle. The abutment surface of the applicator unit firstly has to leave this recess before the transfer into the use position can begin. A second recess, in which the abutment surface of the applicator unit engages when the conveying device is actuated by means of the operating handle, is preferably provided on the operating handle. This second recess thus has the purpose of preventing an actuation-impeding collision during the discharge actuation in the use position.

In particular, an intermediate element, which is described below, can assume the function of the applicator unit abutment surface.

Such an intermediate element is preferably firstly provided for transmitting, when the liquid dispenser is in the use position, the actuation of the operating handle to the conveying device. In particular, the intermediate element can be configured as a pivotable intermediate lever, by means of which the displacement of the operating handle in an actuating direction is transferred into an application of force to the conveying device in a direction that is different from the actuating direction. The intermediate element can in particular deflect, in particular by 90°, an actuation movement that occurs transversely to the piston movement direction.

This intermediate element can form part of the blocking device. For this purpose, the applicator unit abutment surface can be provided on this intermediate element, in particular on a pin portion, by means of which the intermediate element is inserted into a housing of the applicator unit so as to be able to pivot.

A liquid dispenser according to one aspect of the invention can be designed as a disposable dispenser. However, it is preferable for essential parts of the liquid dispenser to be reused. Particularly preferably, in particular a housing of the applicator unit, the protective unit and also the operating handle are reused. These parts that are provided to be reused form a main unit.

In particular, provision can be made for the liquid dispenser to have a replaceable liquid cartridge. In the most straightforward case, this can be formed by a reservoir body, which is coupled to a conveying device that remains in the main unit.

However, the replaceable liquid cartridge preferably comprises, in addition to a reservoir body that defines the liquid reservoir, a discharge nozzle and a conveying device. All of these parts are accordingly exchanged when the liquid cartridge is replaced. The conveying device, which is in particular designed as a pump device or as a valve device, can in such a case be configured in such a manner that it can be actuated by relative movement of the discharge nozzle with respect to the reservoir body.

This can in particular be used by virtue of the discharge nozzle remaining fixed in position on the applicator unit when the liquid cartridge is in the inserted state, whereas the reservoir body can be displaced by means of the operating handle, so that said reservoir body presses against the discharge nozzle and as a result actuates the conveying device, i.e. effects a pumping operation or opens an outlet valve.

The liquid applicator itself is part of the applicator unit, but can be designed as a releasable part. In the inserted state, the liquid applicator is preferably inserted into a cutout of the applicator unit, from which it can be removed without tools. The liquid applicator preferably sits loosely in the cutout, so that it can slide out of the cutout after or during removal of the liquid cartridge. The liquid applicator can thus be exchanged in a straightforward manner. Furthermore, it can itself also be part of the liquid cartridge, in particular by it being fitted onto the discharge nozzle of the liquid cartridge in a clamping manner.

The liquid reservoir and the liquid cartridge are preferably arranged in an interior space of the applicator unit during operation. The exchanging is possible by virtue of a closure flap of the applicator unit being opened. This flap can be provided in particular on a side of the housing that faces away from the liquid applicator or on a longitudinal side.

The liquid dispenser according to one aspect of the invention can be partially provided to be reused in the manner already described. It is therefore considered expedient to combine sets that comprise, in addition to a liquid dispenser including a liquid cartridge, at least one further liquid cartridge. On account of the possible reusability, the invention also relates to a liquid dispenser of the kind described without the liquid cartridge, comprising at least the applicator unit without a liquid reservoir, the protective unit and the operating handle. On account of the reusability, it is considered advantageous for the main unit formed by these components to be robust and high-quality. This means that preferably at least some of the outer surfaces are metallic or else consist of materials other than plastic.

### BRIEF DESCRIPTION OF THE DRAWINGS

Further advantages and aspects of the invention will emerge from the following description of preferred exemplary embodiments of the invention, which are explained below on the basis of the figures.

FIG. 1 shows an overall representation of one exemplary embodiment of a liquid dispenser according to the invention.

FIG. 2 shows main subassemblies of the liquid dispenser in partially isolated form.

FIGS. 3 to 5 show the transfer of the liquid dispenser from a protective state into a use state.

FIG. 6 illustrates the location of installation of a spring as part of a blocking device of the liquid dispenser.

### DETAILED DESCRIPTION

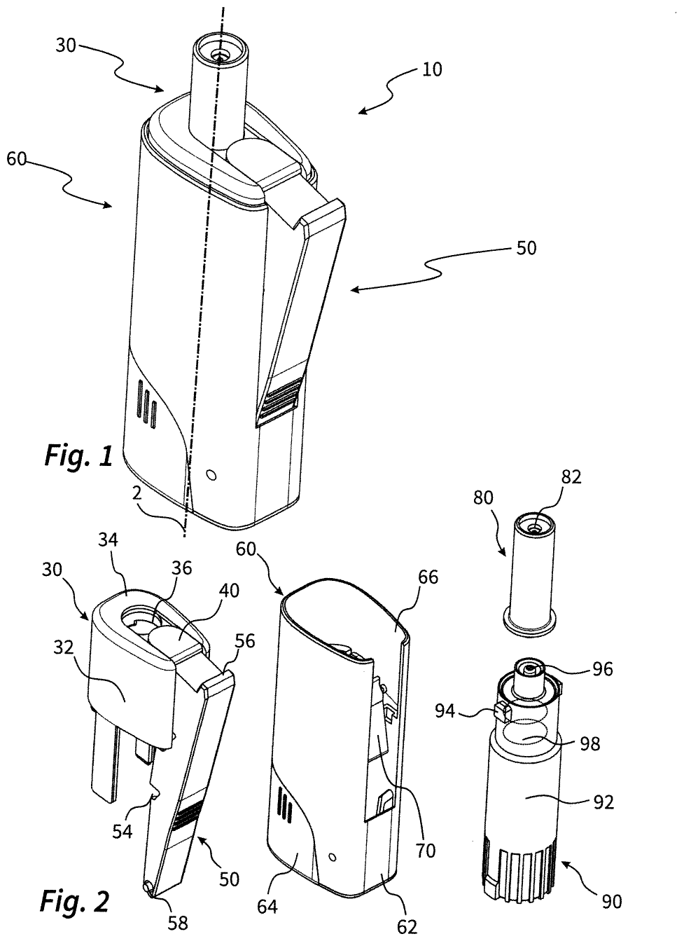

FIG. 1 shows a perspective overall representation of a liquid dispenser 10 according to the invention. As will be explained in more detail below, the liquid dispenser 10 can be brought into a protective state, in which the liquid dispenser, in particular its nozzle unit 82, is protected. From this point, it can be brought into a use state, in which it can be used and liquid can be discharged. FIG. 1 shows the use state.

FIG. 2 shows subassemblies of the liquid dispenser 10 in partially disassembled form.

The liquid dispenser 10 has three primary subassemblies that are essential to the invention and are fundamentally able to move with respect to one another. These subassemblies are those of an applicator unit 60, of a protective unit 30 and of an operating handle 50, which is configured in the manner of an operating lever 50 in the present case. An exchangeable liquid cartridge 90 is provided as a further subassembly. A liquid applicator 80 is likewise represented as a separate subassembly in the present case, but forms part of the applicator unit 60 during operation and can be displaced together with the housing 62 thereof. However, the liquid applicator 80 can be designed as a straightforwardly exchangeable unit.

As can be seen from FIG. 1, the liquid dispenser 10 is configured as a dispenser that is overall elongate and has a slender form, the main direction of extent of which is formed by the longitudinal direction 2. The already mentioned liquid applicator 80 is provided on an upper end side. The operating handle 50 in the form of an operating lever 50 is provided on a longitudinal side.

The liquid dispenser 10 has, in addition to the applicator unit 60, which is represented in the centre of FIG. 2, the abovementioned protective unit 30, which is represented, together with the operating handle 50, on the left in FIG. 2. The protective unit 30 has a protective cover 32 and also an end wall 34. In the use position in FIG. 1, the protective unit 30 penetrates into the applicator unit 60 and as a result unblocks the liquid applicator 80 for use, the latter passing outwards through an aperture 36 in the end wall 34 of the protective unit 30. As can be seen from FIG. 2, a protective panel 40, which can close this aperture 36 in the protective state, is furthermore provided on the protective unit 30.

The operating handle 50 in the form of an operating lever 50 is represented together with the protective unit in FIG. 2. The operating lever 50 is able to move to a limited extent with respect to the protective unit 30. In relation to the longitudinal direction 2, the operating lever 50 is however coupled to the protective unit 30, since its upper end is guided in a slot guide 38 below the end wall 34 of the protective unit 30. However, the operating lever 50 is able to move with respect to the protective unit transversely to the longitudinal direction 2, in order to make the movement sequences that are further explained below possible.

The applicator unit 60 has a housing 62, the interior space of which is provided for receiving the liquid cartridge 90. For this purpose, a detachable flap 64 is provided on the side of the liquid dispenser 10 that faces away from the liquid applicator 80. The liquid cartridge 90 is inserted from below into the interior space and rotated therein, in order to come into engagement with an intermediate lever 70, which can be seen only partially in FIG. 2. In the inserted state, a discharge nozzle 96 of the liquid cartridge 90 bears against the lower end of the liquid applicator 80, so that liquid exiting here flows into the liquid applicator 80. Liquid can be discharged from the liquid cartridge 90 by virtue of a reservoir body 92 of the liquid cartridge 90 being pressed against the discharge nozzle 96 and as a result an internal pump device 98 of the liquid cartridge 90 being actuated. In order to produce this movement, provision is made for the intermediate lever 70, which raises the reservoir body 92 during pivoting via cams 94 on the latter.

The liquid applicator 80 itself is designed, by way of example, as a cylindrical applicator for oral use in the present case. It has a discharge structure 82, which is suitable for atomizing the liquid. In the present case, a nozzle unit 82 in the form of a nozzle plate 82 with a plurality of fine nozzle openings is provided at a distal end of the liquid applicator 80. The liquid is pressed therethrough and atomized in the process.

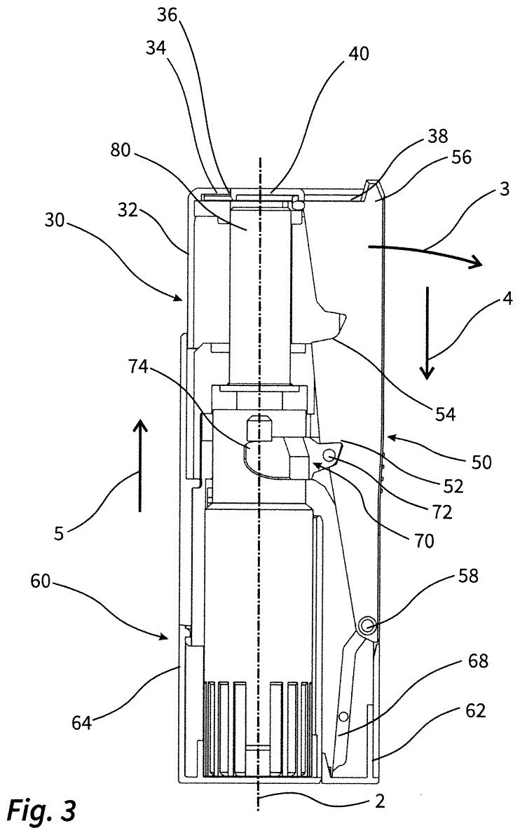

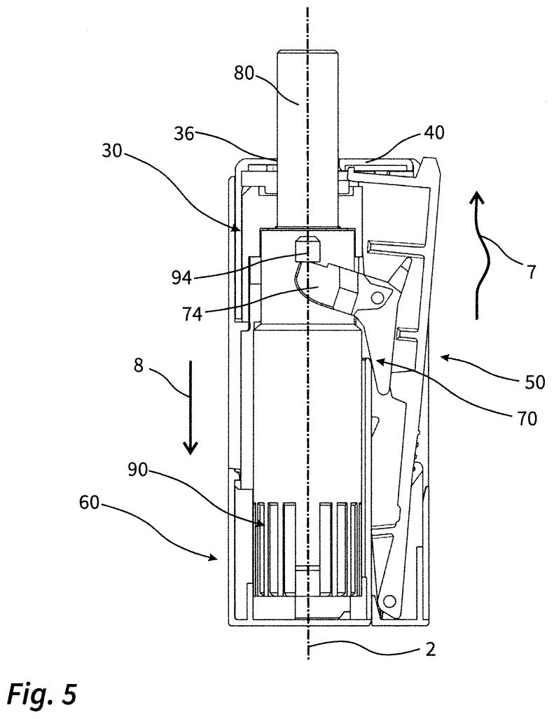

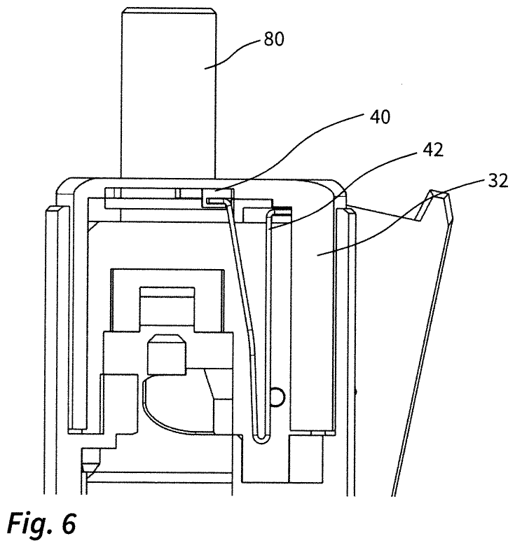

FIGS. 3 to 5 illustrate a sequence, by means of which the liquid dispenser 10 is transferred from a protected state into a usable state, whereby the blocking devices 4, 72, 40 are released and liquid is finally discharged.

FIG. 3 shows the protected state. In this state, the protective unit 30 is in its protective position, i.e. in its upper end position, so that it protrudes above the liquid applicator 80. The liquid applicator 80 is arranged so as to be isolated in an interior space of the protective unit 30. In this state, the aperture 36 of the protective unit 30 is closed by means of the already mentioned protective panel 40, which can move parallel to the end wall 34 of the protective unit and, in the state in FIG. 1, is pressed into the closed position by a spring 42, which is illustrated once again in FIG. 6. In the state in FIG. 3, the operating lever 50 is in an applied position, so that the liquid dispenser 10 has a compact form in relation to the transverse direction.

In this state, it is not possible for the protective unit 30 to be directly displaced with respect to the applicator unit 60, since the abovementioned protective panel 40 provides blocking. In addition, further blocking is provided, specifically by virtue of a first cutout 52 or a pair of first cutouts 52, which is/are in engagement with a bearing region 72 of the intermediate lever, being provided on the inner side of the operating lever 50. An upper side of the bearing region 72 and a side pointing downwards of the operating lever within the abovementioned cutouts 52 form abutment surfaces, which butt against one another and prevent further displacement if direct displacement of the protective unit 30 with respect to the applicator unit 60 is attempted.

In order to use the liquid dispenser 10, the user grips the housing 62 of the applicator unit 60 in such a manner that their thumb rests on the operating lever 50. This is the preferred type of grip.

Using their thumb, the user can then grip a grip elevation 56 on the operating lever 50 and can pivot the latter outwards about the pivot pin 58 in the manner illustrated by the arrow 3. The two blocking devices 40, 52, 72 are released by this movement. The cutout 52 is displaced to the right to such an extent that it disengages from the abutment surface of the intermediate lever 70. The protective panel 40 is pulled to the right by the operating lever 50 against the force of the spring 42, which is represented in FIG. 6, and as a result unblocks the aperture 36.

Once this state has been reached, the user can now pull the operating lever 50, together with the protective unit 30, downwards with respect to the applicator unit. In FIG. 3, this is illustrated by the arrows 4, 5. The common movement is effected by the upper end of the operating lever 50 being guided in the already mentioned slot guide 38 of the protective unit 30.

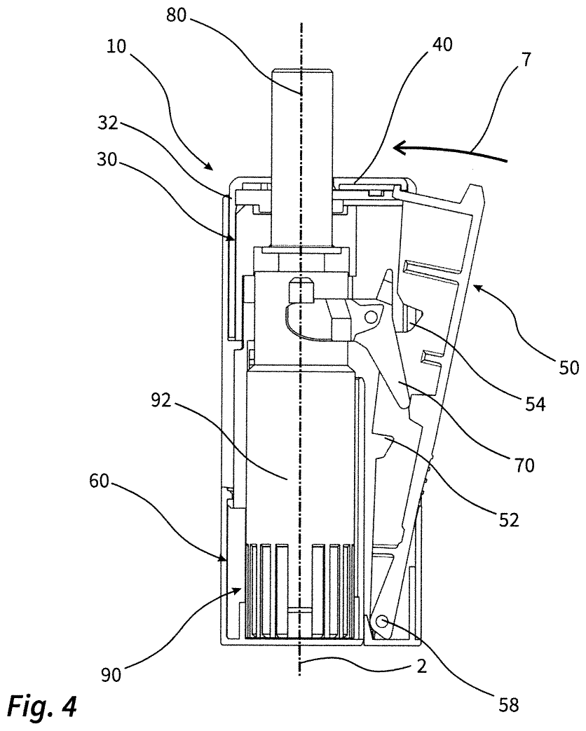

This produces the state in FIG. 4. The protective cover 32 of the protective unit 30 now penetrates into the applicator unit 60 and the liquid applicator 80 assumes an exposed position.

The pivot pin 58 of the operating lever 50 has had to change its relative position in relation to the applicator unit 60 during the movement into the state in FIG. 4. This takes place using a slot guide 68, which is provided on the applicator unit 60. This slot guide 68 has, as can be seen in particular from FIG. 3, a profile that is inclined with respect to the longitudinal direction 2. The distance between the slot guide 68 and the outer wall of the applicator unit 60 increases towards the lower end. This shape results in the lower end of the operating lever 50 penetrating into the housing 62 of the applicator unit 60 during the relative displacement of the protective unit 30 with respect to the applicator unit 60. A separate cutout in the housing 62, through which contamination could enter in the protective state, is therefore not required.

Starting from the state in FIG. 4 and still as part of the same movement sequence of the thumb, the actual actuation of the liquid dispenser 10 is now possible, in order to achieve the discharging of liquid. For this purpose, the operating lever 50 is now pivoted inwards, as illustrated by arrow 6, counter to the previous movement in the direction of the arrow 3. This pivoting movement takes place about the pivot pin 58 of the operating lever, said pivot pin now being arranged in a different position relative to the applicator unit 60, however.

A further cutout 54 or a further pair of cutouts 54 on the operating lever 50 enable(s) the operating lever 50 to move in spite of the intermediate lever 70 and the abutment surfaces thereon. The protective panel 40, which was previously displaced together with the operating lever 50, is not carried along during this movement of the operating lever 50, but rather continues to bear against the liquid applicator 80. For this purpose, the connection between the operating lever 50 and the protective panel 80 is unidirectional. In relation to FIG. 4, the operating lever 50 can thus pull the protective panel 40 to the right, but cannot push it to the left.

As a result of being displaced in the direction of the arrow 6, the operating lever 50 applies torque to the intermediate lever 70, which pivots, so that arms 74 that grip the reservoir body 92 press against the cams 94 from below and as a result raise the reservoir body 92 as a whole. The pump device 98 provided within the liquid cartridge 90 is actuated as a result and causes liquid to be discharged in the form of a fine spray mist by the nozzle unit 82.

After successful discharging, the user presses the applicator unit 60 and the operating lever 50 and thus the protective unit 30 once again in the direction of the starting position, as illustrated by arrows 7 and 8. On account of the abutment surfaces on the intermediate lever 70, the operating lever 50 is again pushed slightly outwards in the process, but then reaches the starting position in FIG. 3 once again. The form of the arrow 7 illustrates the movement profile of the operating lever 50. As soon as the liquid applicator 80 has passed through the aperture 36 into the interior space of the protective unit 30, the protective panel 40 automatically closes on account of the spring force of the spring 42.

The dispenser is now, after one movement, which did not require any regripping at all and enabled guidance from the protective state into the use state, in order to actuate and to discharge liquid and also finally to transfer back, once again in the original protective state.

1. A liquid dispenser, the liquid dispenser comprising:
a liquid reservoir;
a liquid applicator;
a conveying device for conveying liquid from the liquid reservoir to the liquid applicator; and
an applicator unit, the liquid applicator being part of the applicator unit;
a protective unit, the protective unit having a protective cover for protecting the applicator unit,
the protective unit and the applicator unit being displaceable with respect to one another between a protective position and a use position, wherein
in the protective position the protective unit at least partially surrounds the liquid applicator and as a result protects the liquid applicator, and
in the use position the liquid applicator is in an exposed position relative to the protective unit for discharging liquid; and
an operating handle, the operating handle being movable with respect to the applicator unit and with respect to the protective unit,
wherein via the operating handle, the protective unit is displaceable with respect to the applicator unit from the protective position into the use position, and
in the use position the conveying device is actuable and liquid is conveyable from the liquid reservoir to the liquid applicator.

2. The liquid dispenser according to claim 1, wherein
the liquid dispenser has a blocking device, the blocking device preventing the protective unit and the applicator unit from being displaced from the protective position into the use position, and
the blocking device is releasable via the operating handle such that the protective unit and the applicator unit can then be displaced into the use position.

3. The liquid dispenser according to claim 1, wherein
the operating handle is displaceable together with the protective unit with respect to the applicator unit.

4. The liquid dispenser according to claim 1, wherein
a displacement direction of the operating handle is at an angle with respect to a displacement direction of the protective unit and of the applicator unit.

5. The liquid dispenser according to claim 1, wherein
the operating handle comprises a pivotable operating lever.

6. The liquid dispenser according to claim 5, further including a slot guide, and
the pivotable operating lever is guided on the applicator unit by the slot guide.

7. The liquid dispenser according to claim 6, wherein the applicator unit includes a housing,
the slot guide has a direction of extent that, at least in certain sections, deviates from a displacement direction of the protective unit with respect to the applicator unit, so that the operating lever, when the protective unit is transferred from the protective position into the use position, is displaced so as to deviate from the displacement direction in a region of a pivot pin and as a result penetrates at least partially into the housing of the applicator unit.

8. The liquid dispenser according to claim 2, wherein
the blocking device comprises a protective panel, the protective panel being displaceable via the operating handle from a position in aligned arrangement in relation to the liquid applicator into a position that is offset therefrom.

9. The liquid dispenser according to claim 8, wherein
the protective panel is coupled to the operating handle in a unidirectional manner such that the protective panel is displaceable into an open position by the operating handle being moved in an opening direction but the protective panel can remain in the open position if the operating handle is moved in the opposite direction.

10. The liquid dispenser according to claim 8, wherein
the protective panel is assigned a spring arrangement, the spring arrangement constantly applying a force in a direction of a closed position to the protective panel.

11. The liquid dispenser according to claim 2,
the liquid dispenser further comprises an intermediate element, the intermediate element, when the liquid dispenser is in the use position, transmits the actuation of the operating handle to the conveying device.

12. The liquid dispenser according to claim 1, wherein the applicator unit has an outer housing and
the liquid dispenser has a replaceable liquid cartridge,
the liquid applicator being configured as a part that can be released from the outer housing of the applicator unit without tools, wherein in an inserted state, the liquid applicator is inserted into a cutout, from which cutout the liquid applicator is removable without tools and/or is adapted to a discharge nozzle of the liquid cartridge such that said liquid applicator is removable together with the liquid cartridge.

13. The liquid dispenser according to claim 12, wherein
the liquid cartridge has a reservoir body comprising the liquid reservoir,
the liquid cartridge has a discharge nozzle, and
the conveying device is part of the liquid cartridge.

14. The liquid dispenser according to claim 1, wherein
the conveying device is configured as a pump device;
the liquid reservoir is configured as a pressurized reservoir and the conveying device is configured as an outlet valve; or
the liquid applicator comprises a nozzle unit with a plurality of fine nozzle openings for producing an atomized spray jet; or
the liquid reservoir has a volume of less than 50 ml; or
the protective unit or the applicator unit has an outer side, the outer side being at least partially metallic or metallized; or
the protective unit and the applicator unit are movable by at least 5 mm; or
the liquid applicator is configured as a nasal applicator with a conical shape; or
the liquid applicator is configured as a cylindrical oral applicator; or
the protective unit, the applicator unit and the operating handle together form a closed body shape; or
the liquid dispenser comprises a flap configured to permit exchange of a liquid cartridge, the flap being disposed on a side of the liquid dispenser that faces away from the liquid applicator; or
the liquid dispenser is part of a set, the set comprising a liquid cartridge and at least one further liquid cartridge; or
the liquid dispenser has, along a longitudinal axis, a length of at most 120 mm or a length that is longer than a maximum cross section by at least a factor of 2; or
the protective unit is guided within the applicator unit.

15. The liquid dispenser according to claim 1, wherein
the liquid dispenser is configured as a nicotine dispenser for oral application; or
the liquid dispenser is configured as a nasal dispenser, and the liquid applicator is configured as a nasal applicator.

16. The liquid dispenser according to claim 2, wherein:
the blocking device is configured such that the protective unit can only begin to be displaced with respect to the applicator unit once the operating handle has been displaced from a starting position; and/or
the operating handle is coupled to the blocking device such that the blocking device can be released by the operating handle being displaced outwards and in a direction pointing away from the liquid applicator.

17. The liquid dispenser according to claim 3, further including a slot guide, the operating handle being movably guided by the slot guide on the protective unit, the slot guide extending orthogonally to a displacement direction of the protective unit and of the applicator unit.

18. The liquid dispenser according to claim 4, wherein the operating handle is arranged on a first longitudinal side of the liquid dispenser and the liquid dispenser further includes a gripping surface disposed on a second longitudinal side of the liquid dispenser opposite the first longitudinal side, the gripping surface being part of the applicator unit.

19. The liquid dispenser according to claim 5, one of the preceding claims, wherein:
the pivotable operating lever is mounted on the applicator unit at an end of the pivotable operating lever that points away from the liquid applicator and is pivotable about a pivot pin; and/or
the pivotable operating lever is pivotable, starting from a rest position, outwards or away from the liquid applicator to release the blocking device; and/or
the pivotable operating lever is pivotable, starting from a pivoted-out position, inwards or towards the liquid applicator to actuate the conveying device.

20. The liquid dispenser according to claim 8, wherein the protective unit has an end wall with an aperture, the liquid applicator protruding through the aperture in the use position, the aperture being closable by the protective panel.

21. The liquid dispenser according to claim 11, wherein the intermediate element is configured as a pivotable intermediate lever, the displacement of the operating handle in an actuating direction being transferred, via the pivotable intermediate lever, into an application of force to the conveying device in a direction different from the actuating direction.

22. The liquid dispenser according to claim 11, wherein the intermediate element forms part of the blocking device, the protective unit, in the protective position and via the intermediate element, being prevented from moving with respect to the applicator unit.

23. The liquid dispenser according to claim 11, wherein the intermediate element and the operating handle have abutment surfaces, the abutment surfaces, in the protective position, preventing the protective unit from moving with respect to the applicator unit, the operating handle having first and second recesses, the abutment surface of the operating handle being disposed in the first recess of the operating handle, and the abutment surface of the intermediate element engaging in the second recess when the conveying device is actuated by the operating handle.

24. The liquid dispenser according to claim 13, wherein the conveying device is configured such that the conveying device is actuable by relative movement of the discharge nozzle with respect to the reservoir body, the reservoir body being displaceable by the operating handle such that said reservoir body is pressed against the discharge nozzle and actuates the conveying device.
